(19) United States
(12) Patent Application Publication
(10) Pub. No.: US 2022/0163463 A1
(43) Pub. Date: May 26, 2022

(54) APPARATUS, SYSTEM, METHOD, AND STORAGE MEDIUM

(71) Applicant: CANON KABUSHIKI KAISHA, Tokyo (JP)

(72) Inventor: Kazuma Obara, Tokyo (JP)

(21) Appl. No.: 17/521,717

(22) Filed: Nov. 8, 2021

(30) Foreign Application Priority Data
Nov. 20, 2020 (JP) 2020-192934

(51) Int. Cl. G01N 23/04; G06T 5/50; G06F 3/14

(52) U.S. Cl. CPC G01N 23/04; G06T 5/50; G06F 3/14; A61B 6/482; G06T 2207/10116; G01N 2223/401; G06T 2207/20224

(57) ABSTRACT

An apparatus includes a setting unit and an obtaining unit. The setting unit is configured to set an order of transmission for first data and second data, the first data being obtained by using radiation having first energy, the second data being obtained by using radiation having second energy that is lower than the first energy. The obtaining unit is configured to obtain the first data and the second data that have been transmitted in the set order of transmission.

### BACKGROUND OF THE DISCLOSURE

### Field of the Disclosure

The aspect of the embodiments relates to an apparatus, a system, a method, and a storage medium.

### Description of the Related Art

A radiographing apparatus that uses a flat panel detector (FPD) made of a semiconductor material has been widely used for medical imaging diagnosis and non-destructive inspection. One known example of an imaging method using the FPD is a method for obtaining an energy subtraction image, by using a plurality of radiographic images obtained by detecting radiations having different energy components.

An energy subtraction image is generated by performing energy subtraction processing on a plurality of radiographic images that has been obtained by using an FPD. The energy subtraction processing is performed after pieces of radiographic image data have been transmitted from a radiographing apparatus to a control apparatus and the control apparatus has generated radiographic images.

Methods of capturing a plurality of radiographic images includes a method in which one radiographic image is obtained in one imaging, and a method in which two radiographic images are simultaneously obtained in one irradiation as described in a configuration of an FPD discussed in Japanese Patent Application No. 2000-60545.

Energy subtraction imaging is used to make a diagnosis by only using an energy subtraction image or by using both the energy subtraction image and a radiographic image used in subtraction processing. Thus, in energy subtraction imaging, a radiographic image may be checked in some cases before subtraction processing is performed.

However, it may take time to check an image to be used in diagnosis, depending on the order of transmission of radiographic image data.

### SUMMARY OF THE DISCLOSURE

According to an aspect of the embodiments, an apparatus includes a setting unit configured to set an order of transmission for first radiographic image data and second radiographic image data, the first radiographic image data being obtained by using radiation having first energy, the second radiographic image data being obtained by using radiation having second energy that is lower than the first energy; and an obtaining unit configured to obtain the first radiographic image data and the second radiographic image data that have been transmitted in the set order of transmission.

Further features of the disclosure will become apparent from the following description of exemplary embodiments with reference to the attached drawings.

### BRIEF DESCRIPTION OF THE DRAWINGS

FIG. 1 is a block diagram illustrating an example of a radiographing system.

FIG. 2 is a block diagram illustrating a functional configuration example of a radiographing apparatus and a control apparatus according to an exemplary embodiment of the disclosure.

FIG. 3 is a flowchart illustrating a processing procedure performed by a radiographing system according to a first exemplary embodiment.

FIG. 4 is a flowchart illustrating a processing procedure performed by a radiographing system according to a second exemplary embodiment.

FIG. 5 is a timing chart illustrating a processing procedure performed by a radiographing system according to the second exemplary embodiment.

FIG. 6 is a timing chart illustrating a processing procedure performed by a radiographing system according to a third exemplary embodiment.

### DESCRIPTION OF THE EMBODIMENTS

Exemplary embodiments of the disclosure are described below with reference to the attached drawings. The exemplary embodiments described below do not restrict disclosure according to the claims. In addition, not all of the combinations of features described in the exemplary embodiments of the disclosure are essential for solutions of the disclosure.

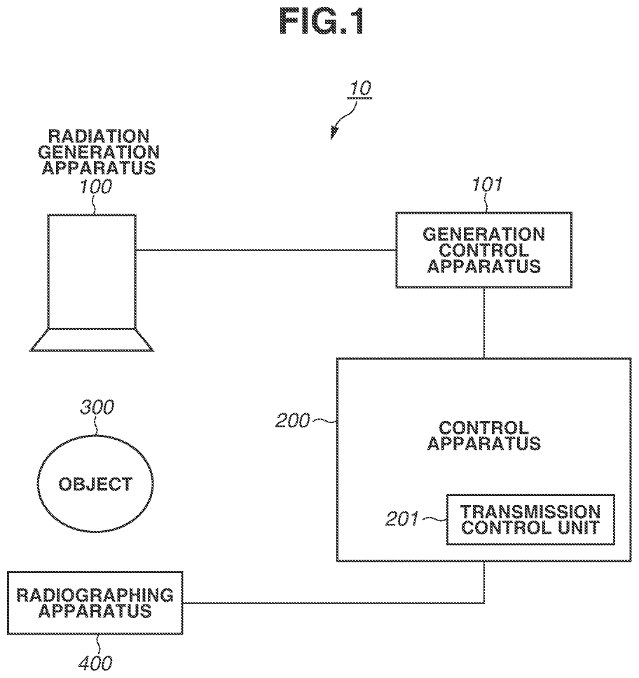

A configuration and an operation of a radiographing system according to exemplary embodiments of the disclosure are described with reference to FIGS. 1 to 3.

FIG. 1 is a diagram illustrating a configuration example of a radiographing system 10 that includes a radiographing apparatus 400 and a control apparatus 200 according to a first exemplary embodiment of the disclosure.

In the present exemplary embodiment, the radiographing system 10 using the control apparatus 200 is a system for obtaining a radiographic image by using energy subtraction. In energy subtraction, an object 300 is imaged plural times by using radiations that are different in energy to obtain a plurality of radiographic images, and the plurality of radiographic images are processed by the control apparatus 200, and thereby a new radiographic image (e.g., a bone image and a soft tissue image) is obtained. In another method, a radiographing apparatus 400 includes a plurality of detectors that captures different levels of radiation energy, and thereby a plurality of radiographic images is obtained with single irradiation.

The radiographing system 10 electrically captures an optical image that has been converted from a radiation incident on the radiographing apparatus 400, and obtains radiographic image data for generating a radiographic image. The radiographing system 10 includes the radiographing apparatus 400, a radiation generation apparatus 100 configured to emit radiation, a generation control apparatus 101 configured to control the radiation generation apparatus 100, and the control apparatus 200 configured to control the generation control apparatus 101 and the radiographing apparatus 400.

The control apparatus 200 may include a computer (a processor) and a memory that stores a program to be provided to the computer. The control apparatus 200 further includes a transmission control unit 201 that controls radiographic image data to be transmitted from the radiographing apparatus 400. The transmission control unit 201 may include a part of the program stored in the memory of the control apparatus 200. The transmission control unit 201 may also be disposed independently of the control apparatus 200, and may include a computer (a processor) and a memory that stores a program to be provided to the computer. The entirety or part of the control apparatus 200 may include a digital signal processor (DSP) or a programmable logic array (PLA). The control apparatus 200 and the transmission control unit 201 may be designed and manufactured by using a logic synthesis tool based on a file describing operations of the control apparatus 200 and the transmission control unit 201. The control apparatus 200 may also function as a user interface of the radiographing system 10. In such a case, the control apparatus 200 may include, for example, an input unit to which a user inputs conditions of imaging for obtaining a radiographic image, and a display unit, such as a display, with which the user checks input information.

The generation control apparatus 101 controls radiation irradiation of the radiation generation apparatus 100. The generation control apparatus 101 may include, for example, an exposure switch. When a user turns on the exposure switch, the generation control apparatus 101 may cause the radiation generation apparatus 100 to emit radiation, and may report, to the control apparatus 200, information indicating a timing when radiation is emitted. The generation control apparatus 101 may also cause the radiation generation apparatus 100 to emit radiation in accordance with a command from the control apparatus 200.

The radiation generation apparatus 100 has a function of changing energy (a wavelength) of radiation. The radiation generation apparatus 100 can change the energy of radiation by changing, for example, tube voltage (a voltage to be applied between a cathode and an anode of the radiation generation apparatus 100) under the control of the generation control apparatus 101. The radiation generation apparatus 100 can emit radiations having a plurality of energy values that is different from each other.

In the configuration example illustrated in FIG. 1, the radiographing apparatus 400 and the control apparatus 200 are disposed independently of each other. However, all or some of the functions of the control apparatus 200 may be incorporated into the radiographing apparatus 400. Some of the functions of the radiographing apparatus 400 may also be incorporated into the control apparatus 200.

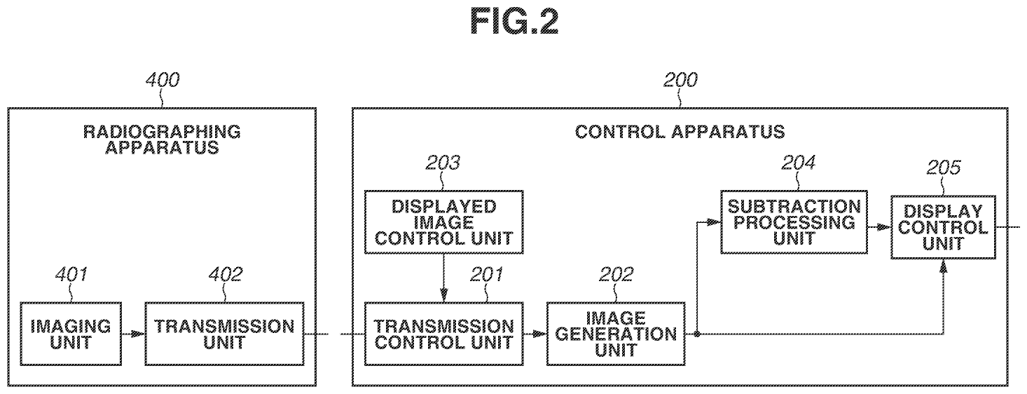

FIG. 2 is a block diagram illustrating functional configuration examples of a radiographing apparatus and a control apparatus according to an exemplary embodiment of the disclosure.

The control apparatus 200 includes the transmission control unit 201, an image generation unit 202, a displayed image control unit 203, a subtraction processing unit 204, and a display control unit 205. An operation of each processing unit is performed and controlled by the processor or the like of the control apparatus 200 illustrated in FIG. 1.

The transmission control unit 201 controls radiographic image data to be transmitted by the radiographing apparatus 400. The transmission control unit 201 controls, for example, at least either the order of transmission of radiographic image data or a data size of radiographic image data to be transmitted. The transmitted radiographic image data is output to the image generation unit 202.

The image generation unit 202 outputs (generates) a radiographic image based on the radiographic image data. In a case where energy subtraction is not performed, the radiographic image is output to the display control unit 205.

In contrast, in a case where energy subtraction is performed, the radiographic image is output to the subtraction processing unit 204.

The displayed image control unit 203 outputs the order of control to the transmission control unit 201 in accordance with stored setting of the order of display for a plurality of radiographic images and an energy subtraction image.

The subtraction processing unit 204 performs energy subtraction processing by using the plurality of radiographic images. A generated energy subtraction image is output to the display control unit 205.

The display control unit 205 displays, on the display unit (not illustrated), at least either a radiographic image or an energy subtraction image that has been input from the image generation unit 202 or the subtraction processing unit 204. The display unit is a device configured to display various types of information generated by the control apparatus 200, and typically, a liquid crystal display is used. However, the display unit may also be a display of another scheme, such as a plasma display, an organic electroluminescence (EL) display, or a field emission display (FED).

The radiographing apparatus 400 includes an imaging unit 401 and a transmission unit 402. An operation of each processing unit is performed and controlled by a processor or the like of the radiographing apparatus 400 illustrated in FIG. 1.

The imaging unit 401 detects emitted radiation as electric charges, and performs analog-to-digital (A/D) conversion on the detected electric charges to obtain radiographic image data. The obtained radiographic image data is output to the transmission unit 402.

The transmission unit 402 transmits radiographic image data for generating a radiographic image from the radiographing apparatus 400 to the control apparatus 200. The transmission unit 402 also changes the order of transmission or a size of image data to be transmitted based on the control of the transmission control unit 201, and transmits the radiographic image data.

Energy subtraction imaging is used to make a diagnosis not only by using an energy subtraction image but also by using both the energy subtraction image and a radiographic image used in subtraction processing. Thus, in energy subtraction imaging, a radiographic image before subtraction processing is to be checked.

On the other hand, at least two or more radiographic images are captured before subtraction processing. In many cases, only any of the captured radiographic images is used in diagnosis, and in some cases, the other captured radiographic images are not used in the diagnosis for the subtraction processing. Accordingly, a radiographic image to be used in diagnosis is transmitted with priority, and this can thereby reduce a waiting time before a radiographic image is checked.

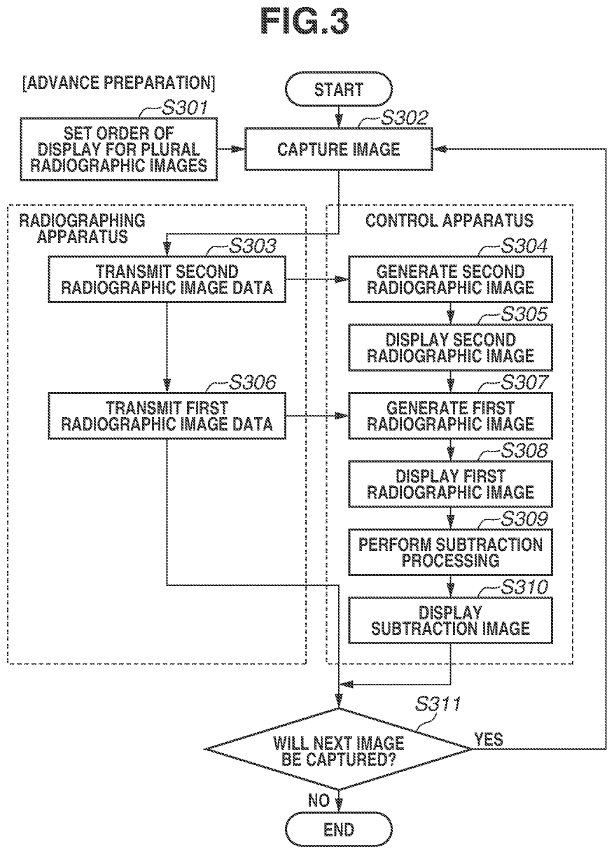

A processing procedure performed by a radiographing system according to the first exemplary embodiment is described with reference to the flowchart illustrated in FIG. 3. An example here obtains two radiographic images as a plurality of radiographic images. The two radiographic images are respectively referred to as a first radiographic image and a second radiographic image in the order of imaging. In energy subtraction processing, at least a plurality of radiographic images is used, and therefore three or more radiographic images may be used.

### (Step S301: Setting Order of Display for Plural Radiographic Images)

In step S301, the displayed image control unit 203 sets the order of display for a plurality of radiographic images and an energy subtraction image.

The order of display may be any order input by a user, or may be set in advance. In a case where the order of display is set in advance, for example, correspondence between a diagnostic purpose and an image to be used with priority for the diagnostic purpose may be set in advance, and the diagnostic purpose may be selected, and thereby the order of display may be set. More specifically, for example, in a case where a diagnosis is made by using a subtraction image with a bone part emphasized, an imaging diagnosis is made by also using an image captured with low energy. In the case described above, the order is set in such a way that radiographic image data obtained with low energy is transmitted and displayed with priority. In contrast, in a case where a diagnosis is made by using an energy subtraction image with a bone part removed and soft tissue emphasized, an imaging diagnosis is made by also using an image captured with high energy. In the case described above, the order is set in such a way that radiographic image data obtained with high energy is transmitted and displayed with priority. Stated another way, in a case where a diagnostic purpose is the observation of soft tissue, the displayed image control unit 203 performs setting to give priority to the order of transmission of first radiographic image data over the order of transmission of second radiographic image data. The first radiographic image data has been obtained by using radiation having first energy. The second radiographic image data has been obtained by using radiation having second energy that is lower than the first energy. In a case where a diagnostic purpose is the observation of a bone part, setting is performed to give priority to the order of transmission of the second radiographic image data over the order of transmission of the first radiographic image data.

In the description above, the order of display has been set. However, the order of display does not always need to be set for each image, and any configuration where the order of display is controlled is sufficient. A configuration may be employed where the order of transmission is set. In such a case, for example, a display is conducted on the display unit in the same order as the order of transmission. Stated another way, the displayed image control unit 203 corresponds to an example of a setting unit that sets the order of transmission for a first radiographic image and a second radiographic image. The order of display may be set after the order of transmission has been set. Furthermore, whether each image will be displayed may be set after the order of transmission has been set.

In the steps that follow, an example is described where setting has been performed in such a way that a second radiographic image serves as an image to be used in imaging diagnosis together with an energy subtraction image, and the second radiographic image is transmitted with higher priority than a first radiographic image.

### (Step S302: Capturing Image)

In step S302, the radiographing apparatus 400 captures a radiographic image. Radiographic image data obtained in imaging is retained by the radiographing apparatus 400. The displayed image control unit 203 controls the transmission control unit 201 to display a second radiographic image with higher priority than a first radiographic image.

### (Step S303: Transmitting Second Radiographic Image Data)

In step S303, the transmission unit 402 transmits, to the control apparatus 200, second radiographic image data for generating the second radiographic image, under the control of the transmission control unit 201. More specifically, the transmission control unit 201 transmits, to the transmission unit 402, the order of transmission of radiographic image data in accordance with the order of transmission that has been set by the displayed image control unit 203 and has been output to the transmission control unit 201. The transmission unit 402 then transmits radiographic image data in accordance with the order of transmission that has been output from the transmission control unit 201.

### (Step S304: Generating Second Radiographic Image)

In step S304, the image generation unit 202 generates the second radiographic image from the second radiographic image data. The image generation unit 202 outputs the generated second radiographic image to the display control unit 205.

### (Step S305: Displaying Second Radiographic Image)

In step S305, the display control unit 205 displays the second radiographic image on the display unit.

### (Step S306: Transmitting First Radiographic Image Data)

In step S306, the transmission unit 402 transmits, to the control apparatus 200, first radiographic image data for generating the first radiographic image, under the control of the transmission control unit 201.

### (Step S307: Generating First Radiographic Image)

In step S307, the image generation unit 202 generates the first radiographic image from the first radiographic image data. The image generation unit 202 outputs the generated first radiographic image to the display control unit 205.

### (Step S308: Displaying First Radiographic Image)

In step S308, the display control unit 205 displays the first radiographic image on the display unit.

### (Step S309: Performing Subtraction Processing)

In step S309, the subtraction processing unit 204 performs energy subtraction processing by using the first radiographic image and the second radiographic image that have been generated by the image generation unit 202, and generates an energy subtraction image.

### (Step S310: Displaying Subtraction Image)

In step S310, the display control unit 205 displays, on the display unit, the energy subtraction image generated by the subtraction processing unit 204.

### (Step S311: Will Next Image be Captured?)

Finally, in step S311, it is determined whether a next image will be captured? In a case where a next image will be captured (YES in step S311), the processing proceeds to step S301. In a case where a next image will not be captured (NO in step S311), the processing is terminated.

By doing the above, the processing of the radiographing system is performed.

As described above, a radiographing system according to the present exemplary embodiment can effectively transmit a plurality of radiographic images in energy subtraction imaging.

Furthermore, a radiographic image to be used in diagnosis is transmitted with priority in accordance with the order of transmission that has been set before imaging, and the radiographic image is displayed. This enables a reduction in a waiting time before a desired image is checked.

In the present exemplary embodiment, the display control unit 205 displays all of the captured radiographic images on the display unit, but radiographic images other than a radiographic image transmitted with priority do not always need to be displayed. Stated another way, the displayed image control unit 203 may set the non-display of a radiographic image to be used to generate an energy subtraction image. For example, non-display may be set by receiving an input from a user. Alternatively, non-display may be automatically set for a radiographic image that is different from a radiographic image that has been set to be displayed with priority.

The present exemplary embodiment has described an example in a case where irradiation is performed plural times with different energy and a plurality of radiographic images are captured. However, the present exemplary embodiment may also be employed in a case where radiographic images are captured in single irradiation using a radiographing apparatus including a plurality of detectors that detects different energy. Stated another way, in the case of use of a radiographing apparatus in which the order of transmission is not determined according to the order of imaging, similarly, the order of transmission may be controlled according to setting of the displayed image control unit 203.

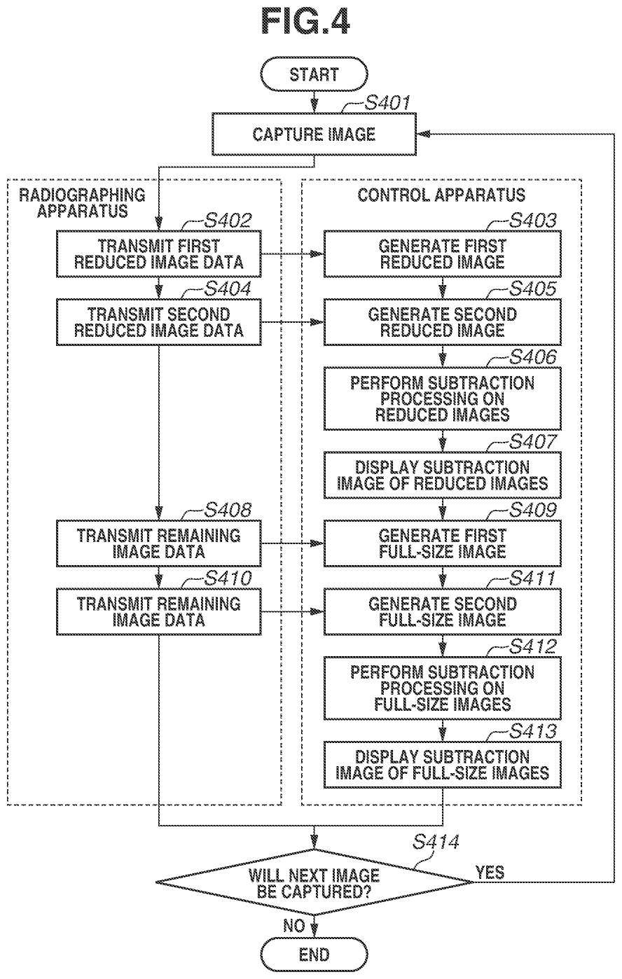

A processing procedure performed by a radiographing system according to a second exemplary embodiment is described with reference to the flowchart illustrated in FIG. 4. Here, an example of capturing two radiographic images as a plurality of radiographic images is described. The two radiographic images are respectively referred to as a first radiographic image and a second radiographic image. In energy subtraction processing, at least a plurality of radiographic images is used, and therefore three or more radiographic images may be used.

The present exemplary embodiment describes an example of separately transmitting two pieces of radiographic image data, which are full-size image data and reduced image data that has been obtained from a full-size image data and has a reduced image data size. It is sufficient if an image that is smaller in size than a full-size image is initially transmitted, and transmission may be separately performed three times or more.

### (Step S401: Capturing Image)

In step S401, the radiographing apparatus 400 captures a radiographic image.

Image data obtained in imaging is retained by the radiographing apparatus 400. The displayed image control unit 203 controls the transmission control unit 201 to display a subtraction image of reduced images as a first priority displayed image and display a subtraction image of full-size images as a second priority displayed image.

### (Step S402: Transmitting First Reduced Image Data)

In step S402, the transmission unit 402 transmits, to the control apparatus 200, first reduced image data to be used to generate a subtraction image of reduced images that serves as the first priority displayed image, under the control of the transmission control unit 201.

### (Step S403: Generating First Reduced Image)

In step S403, the image generation unit 202 generates a first reduced image from the first reduced image data. The image generation unit 202 outputs the generated first reduced image to the subtraction processing unit 204.

### (Step S404: Transmitting Second Reduced Image Data)

In step S404, the transmission unit 402 transmits, to the control apparatus 200, second reduced image data to be used to generate a subtraction image of reduced images that serves as the first priority displayed image, under the control of the transmission control unit 201.

### (Step S405: Generating Second Reduced Image)

In step S405, the image generation unit 202 generates a second reduced image from the second reduced image data. The image generation unit 202 outputs the generated second reduced image to the subtraction processing unit 204.

### (Step S406: Performing Subtraction Processing on Reduced Images)

In step S406, the subtraction processing unit 204 performs energy subtraction processing by using the first and the second reduced images that have been generated by the image generation unit 202, and generates an energy subtraction image. The subtraction processing unit 204 outputs the generated energy subtraction image to the display control unit 205.

### (Step S407: Displaying Subtraction Image of Reduced Images)

In step S407, the display control unit 205 displays, on the display unit, the energy subtraction image generated by the subtraction processing unit 204.

### (Step S408: Transmitting First Full-Size Image Data)

In step S408, the transmission unit 402 transmits remaining image data that is used to generate a first full-size image and has not yet been transmitted in step S402, under the control of the transmission control unit 201. The transmission unit 402 may also transmit first full-size image data. In such a case, step S408 is skipped, and the first full-size image data is output to the transmission control unit 201.

### (Step S409: Generating First Full-Size Image)

In step S409, the image generation unit 202 synthesizes the remaining data that has been transmitted in step S408 with the first reduced image data, and generates the first full-size image. The image generation unit 202 outputs the generated first full-size image to the display control unit 205.

### (Step S410: Transmitting Second Full-Size Image Data)

In step S410, the transmission unit 402 transmits remaining image data that is used to generate a second full-size image and has not yet been transmitted in step S402, under the control of the transmission control unit 201. The transmission unit 402 may transmit second full-size image data. In such a case, step S411 is skipped, and the second full-size image data is output to the transmission control unit 201.

### (Step S411: Generating Second Full-Size Image)

In step S411, the image generation unit 202 synthesizes the remaining data that has been transmitted in step S410 with the second reduced image data, and generates the second full-size image. The image generation unit 202 outputs the generated second full-size image to the display control unit 205.

### (Step S412: Performing Subtraction Processing on Full-Size Images)

In step S412, the subtraction processing unit 204 performs energy subtraction processing by using the first full-size image and the second full-size image that have been generated by the image generation unit 202, and generates an energy subtraction image.

### (Step S413: Displaying Subtraction Image of Full-Size Images)

In step S413, the display control unit 205 displays, on the display unit, the energy subtraction image generated by the subtraction processing unit 204.

### (Step S414: Will Next Image Be Captured?)

Finally, in step S414, it is determined whether a next image will be captured? In a case where a next image will be captured (YES in step S414), the processing proceeds to step S401.

By doing the above, the processing of the radiographing system is performed.

By doing the above, a transmission size of a plurality of radiographic images to be used in energy subtraction processing is controlled. This enables an energy subtraction image of reduced images to be checked prior to the display of an energy subtraction image of full-size images. A user can thereby check an image in a shorter waiting time than is conventional.

### First Variation Example

A first variation example describes an example in a case referred to as one-shot energy subtraction imaging where a radiographing apparatus 400 includes a plurality of detectors that detects radiations that are different in energy, and the radiographing apparatus 400 is used to obtain a plurality of radiographic images in one imaging, in the second exemplary embodiment.

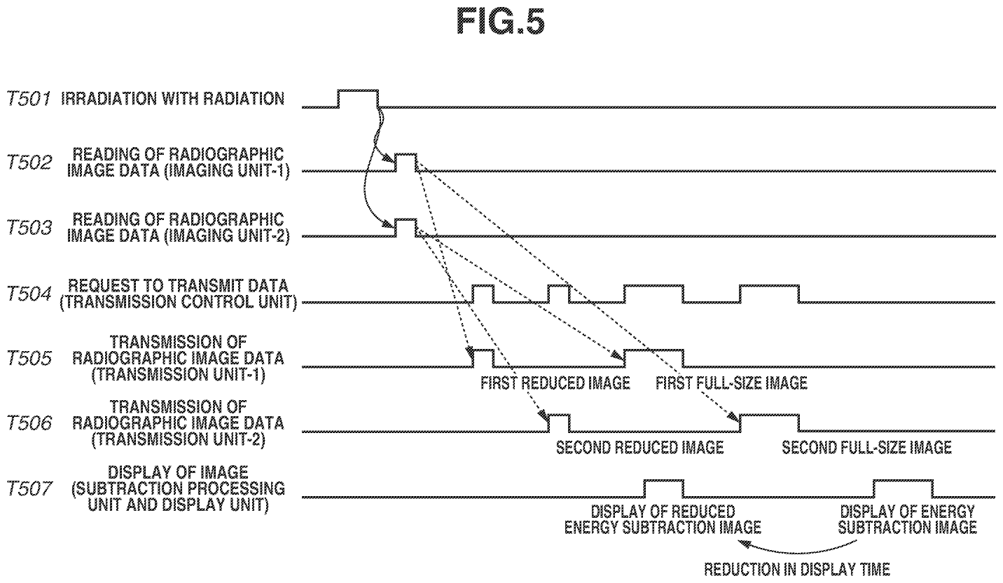

A processing procedure performed by a radiographing system is described with reference to the timing chart illustrated in FIG. 5.

T501 denotes a signal indicating a state of irradiation of radiation. T502 and T503 denote signals respectively indicating states where the imaging unit 401 reads respective plural radiographic images. T504 denotes a signal indicating a request from the transmission control unit 201 for the transmission unit 402 to transmit data. T505 and T506 denote signals each indicating an image transmission time point of the transmission unit 402. T507 denotes a signal indicating a time point at which the display control unit 205 displays an image after energy subtraction processing is performed.

During an irradiation period, the imaging unit 401 is in an accumulation state, and the imaging unit 401 detects radiation to obtain an image signal. Such an image signal (a radiographic image signal) includes a signal component obtained by detecting radiation and a dark component generated by a photoelectric conversion element. Such an image signal is read from a pixel array, and digital image data is obtained.

The transmission unit 402 performs, for example, thinning-out processing or addition processing, and generates reduced image data having a small amount of data from a radiographic image. The transmission unit 402 transmits the reduced image data to the control apparatus 200.

After the completion of processing of transmitting the reduced image data, a transmission control unit 201 according to an exemplary embodiment causes the transmission of radiographic image data (full-size image data) serving as the basis of the reduced image data. In a case where image processing including addition processing is performed in generating a preview image and in a case where original image data fails to be restored from the preview image, in one embodiment, full-size image data is to be transmitted.

The control apparatus 200 performs energy subtraction processing by using reduced images generated from plural pieces of reduced image data that have been generated. The display control unit 205 then displays, on the display unit, an energy subtraction image of the reduced images.

The control apparatus 200 performs energy subtraction processing by using a plurality of fill-size images that has been generated. The display control unit 205 then displays, on the display unit, an energy subtraction image of the full-size images instead of the energy subtraction image of the reduced images.

The present variation example has described an example where the display control unit 205 only displays an energy subtraction image. However, the first reduced image, the second reduced image, the first full-size image, or the second full-size image that has been transmitted may be appropriately displayed in the signal of T507.

### Second Variation Example

A second variation example describes an example in a case where a plurality of radiographic images is obtained by a radiographing apparatus 400 including a single detector is used, which is referred to as two-shot energy subtraction imaging, in the second exemplary embodiment. The plurality of radiographic images are obtained by performing plural times of imaging by using radiations having different energies.

Differences from the first variation example will be mainly described. A configuration of a radiographing system according to the present exemplary embodiment is similar to the configuration described with reference to FIG. 1.

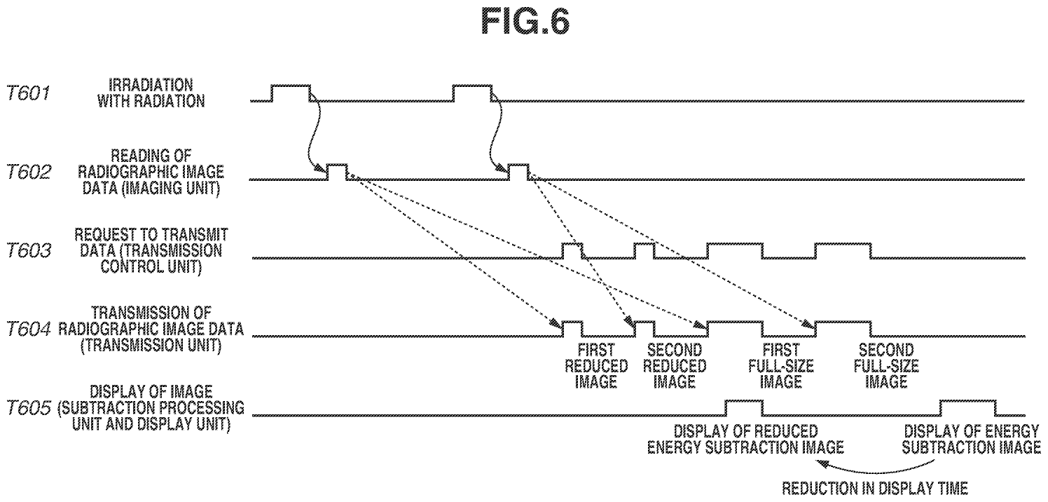

FIG. 6 is a timing chart illustrating a processing procedure performed by a radiographing system according to a third exemplary embodiment.

T601 denotes a signal indicating a state of irradiation with radiation. T602 denotes a signal indicating a state where the imaging unit 401 reads an image in each irradiation. T603 denotes a signal indicating a request from the transmission control unit 201 for the transmission unit 402 to transmit data. T604 denotes a signal indicating an image transmission time point of the transmission unit 402. T605 denotes a time point at which the display control unit 205 displays an image after energy subtraction processing is performed.

During an irradiation period, the imaging unit 401 is in an accumulation state, and the imaging unit 401 detects radiation to obtain an image signal. Such an image signal (a radiographic image signal) includes a signal component obtained by detecting radiation and a dark component generated by a photoelectric conversion element. Such an image signal is read from a pixel array, and digital radiographic image data is obtained. In the present configuration, irradiation is performed plural times by using radiations of different energies: for example, first irradiation is performed to obtain an image by high-energy, and second irradiation is performed to obtain an image by low-energy.

The transmission unit 402 performs, for example, thinning-out processing, or addition processing, and generates reduced image data having a smaller amount of data from radiographic image data. The transmission unit 402 transmits the reduced image data to the control apparatus 200.

After the completion of processing for transmitting all pieces of reduced image data, the transmission unit 402 transmits all pieces of radiographic image data (full-size images) serving as the basis of all pieces of reduced image data. In a case where image processing including addition processing is performed in generating a preview image and in a case where original image data fails to be restored from the preview image, in one embodiment, a full-size image is to be transmitted.

Processes that follow, a display of an energy subtraction image using reduced images, transmission of full-size images, and a display of an energy subtraction image are similar to processes in the second exemplary embodiment.

As described above, a radiographing system according to the present exemplary embodiment enables a user to check an energy subtraction image in a shorter waiting time than is conventional, even in a case where a plurality of radiographic images is obtained in plural times of imaging using radiations that are different in energy, similarly to the first exemplary embodiment.

The present variation example has described an example where the display control unit 205 only displays an energy subtraction image. However, the first reduced image, the second reduced image, the first full-size image, or the second full-size image that has been transmitted may be appropriately displayed, in the signal of T605.

### Third Variation Example

As a method for generating a reduced image to be transmitted in the second, and the third exemplary embodiments, a technique for extracting a region of interest that has been arbitrarily designated before imaging or a technique of a radiographing apparatus mounted with an auto exposure control (AEC) mechanism for extracting an AEC region of recognition is also applicable in addition to thinning-out processing or addition processing.

These techniques can reduce a waiting time before display while a reduction in image quality due to thinning-out processing is avoided, in a case where a diagnosis objective region can be determined in advance.

As described above, according to an exemplary embodiment of the disclosure, a plurality of radiographic images can be effectively transmitted in energy subtraction imaging.

### Other Embodiments

Embodiment(s) of the disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2020-192934, filed Nov. 20, 2020, which is hereby incorporated by reference herein in its entirety.

What is claimed is:

1. An apparatus comprising:
a setting unit configured to set an order of transmission for first data and second data, the first data being obtained by using radiation having first energy, the second data being obtained by using radiation having second energy that is lower than the first energy; and
an obtaining unit configured to obtain the first data and the second data that have been transmitted in the set order of transmission.

2. The apparatus according to claim 1, further comprising:
a generation unit configured to generate a first image from the first data and generate a second image from the second data; and
a display control unit configured to display, on a display unit, at least any image of the first image and the second image, and an energy image obtained by performing subtraction processing on the first image and the second image.

3. The apparatus according to claim 2, wherein the display control unit displays, on the display unit, at least a radiographic image generated from data that has been transmitted earlier from among the first image and the second image, prior to the energy image.

4. The apparatus according to claim 1, wherein the setting unit sets the order of transmission such that the first data is transmitted with higher priority than the second data in a case where an energy image with soft tissue emphasized is generated, and the second data is transmitted with higher priority than the first data in a case where an energy image with a bone part emphasized is generated.

5. The apparatus according to claim 1, further comprising a control unit configured to control the order of transmission for the first data and the second data in accordance with the order of set transmission.

6. The apparatus according to claim 1,
wherein the setting unit receives selection of a diagnostic purpose of the energy image, and
wherein the setting unit sets the order of transmission for the first data and the second data based on the selected diagnostic purpose.

7. The apparatus according to claim 6,
wherein in a case where the diagnostic purpose is observation of soft tissue, the setting unit performs setting to give priority to the order of transmission of the first data over the order of transmission of the second data, and
wherein in a case where the diagnostic purpose is observation of a bone part, the setting unit performs setting to give priority to the order of transmission of the second data over the order of transmission of the first data.

8. The apparatus according to claim 5, wherein the setting unit sets whether to display, on the display unit, at least any radiographic image of the first image and the second image.

9. The apparatus according to claim 1, wherein the setting unit controls the order of transmission such that first reduced data and second reduced data are transmitted prior to the first data and the second data, the first reduced data being generated from the first data and having a data size that is smaller than a data size of the first data, the second reduced data being generated from the second data and having a data size that is smaller than a data size of the second data.

10. The apparatus according to claim 9,
wherein the first reduced data and the second reduced data are generated by extracting an arbitrary region of interest from the first data and the second data, and
wherein the setting unit sets the order of transmission for the first reduced data and the second reduced data that have been generated by extracting the arbitrary region of interest.

11. The apparatus according to claim 9,
wherein the first reduced data and the second reduced data are generated by performing thinning-out processing on the first data and the second data, and
wherein the setting unit sets the order of transmission for the first reduced data and the second reduced data that have been generated by performing the thinning-out processing.

12. The apparatus according to claim 9,
wherein the first reduced data and the second reduced data are generated by extracting an auto exposure control (AEC) region of recognition from the first data and the second data, and
wherein the setting unit sets the order of transmission for the first reduced data and the second reduced data that have been generated by extracting the AEC region of recognition.

13. An apparatus comprising:
a setting unit configured to set an order of display for at least any image of a first image and a second image, and an energy image, the first image being obtained by using radiation having first energy, the second image being obtained by using radiation having second energy that is lower than the first energy, the energy image being obtained by performing subtraction processing on the first image and the second image; and
a display control unit configured to display, on a display unit, at least any image of the first image and the second image, and the energy image in the set order of display.

14. A system comprising:
a radiographing apparatus; and
a control apparatus,
wherein the radiographing apparatus includes:
an obtaining unit configured to obtain first data by detecting radiation having first energy and obtain second data by detecting radiation having second energy that is lower than the first energy; and
a transmission unit configured to transmit the first data and the second data to the control apparatus, and
wherein the control apparatus includes:
a setting unit configured to set an order of transmission for the first data and the second data that are transmitted from the radiographing apparatus.

15. A method comprising:
setting an order of transmission for first data and second data, the first data being obtained by using radiation having first energy, the second data being obtained by using radiation having second energy that is lower than the first energy; and
obtaining the first data and the second data that have been transmitted in the set order of transmission.

16. A method comprising:
setting an order of transmission for first data, first reduced data, second data, and second reduced data, the first data being obtained by using radiation having first energy, the first image data being generated from the first data and having a data size that is smaller than a data size of the first data, the second data being obtained by using radiation having second energy that is lower than the first energy, the second reduced data being generated from the second data and having a data size that is smaller than a data size of the second data;
first transmitting the first reduced data and the second reduced data prior to the first data and the second data;
displaying, on a display unit, a first energy image generated by using the first reduced data and the second reduced data;
second transmitting the first data and the second data; and
displaying, on the display unit, a second energy image generated by using the first data and the second data.

17. A storage medium that stores a program that causes a computer to perform a method comprising:
setting an order of transmission for first data and second data, the first data being obtained by using radiation having first energy, the second data being obtained by using radiation having second energy that is lower than the first energy; and
obtaining the first data and the second data that have been transmitted in the set order of transmission.

18. The storage medium according to claim 17, further comprising:
generating a first radiographic image from the first radiographic image data and generate a second radiographic image from the second radiographic image data; and
displaying, on a display unit, at least any radiographic image of the first radiographic image and the second radiographic image, and an energy image obtained by performing subtraction processing on the first radiographic image and the second radiographic image.

19. The storage medium according to claim 17, wherein the setting sets the order of transmission such that the first data is transmitted with higher priority than the second data in a case where an energy image with soft tissue emphasized is generated, and the second data is transmitted with higher priority than the first data in a case where an energy image with a bone part emphasized is generated.

20. The storage medium according to claim 17, further comprising controlling the order of transmission for the first data and the second data in accordance with the order of set transmission.
